(19) United States
(12) Patent Application Publication
(10) Pub. No.: US 2026/0364307 A1
(43) Pub. Date: Nov. 19, 2026

(54) Intermittent-Catheter Assemblies and Methods Thereof

(71) Applicant: C. R. Bard, Inc., Franklin Lakes, NJ (US)

(72) Inventors: James David Hughett, SR., Monroe, GA (US); Ronald N. Legaspi, Alpharetta, GA (US); Kyle Daw, Smyrna, GA (US); Steven Michael Breen, Decatur, GA (US)

(21) Appl. No.: 19/753,763

(22) Filed: Jul. 24, 2026

Related U.S. Application Data
(62) Division of application No. 18/019,647, filed on Jul. 30, 2021, now Pat. No. 12,691,250.
(60) Provisional application No. 63/060,627, filed on Aug. 3, 2020.

(51) Int. Cl. A61M 25/00

(52) U.S. Cl. CPC A61M 25/002; A61M 25/0043; A61M 2025/0046; A61M 2025/006

(57) ABSTRACT

Intermittent-catheter assemblies and packages, carrying cases, and methods thereof. An intermittent-catheter assembly can include an intermittent catheter and a catheter housing. The intermittent catheter can include a proximal piece, a cap configured to cap a proximal opening of the proximal piece, and a catheter tube fluidly connected to the proximal piece. The catheter housing can be around at least the catheter tube. The catheter housing can include a distal piece and a collapsible sheath. The distal piece can include a lubricating means for lubricating the catheter tube when the distal piece is proximally slid over the catheter tube. The collapsible sheath can include a distal portion coupled to the distal piece and a proximal portion coupled to a neck of the proximal piece. An entirety of the catheter tube can be disposed in the catheter housing in a packaged state of the intermittent-catheter assembly.

### PRIORITY

This application is a division of U.S. patent application Ser. No. 18/019,647, filed Feb. 3, 2023, now U.S. Pat. No. 12,691,250, which is a U.S. national stage of International Application No. PCT/US2021/044021, filed Jul. 30, 2021, which claims the benefit of priority to U.S. Provisional Application No. 63/060,627, filed Aug. 3, 2020, each of which is incorporated by reference in its entirety into this application.

### BACKGROUND

Users of urinary catheters such as intermittent catheters self-catheterize four to six times a day. However, catheterization with such intermittent catheters can be messy. Indeed, existing intermittent catheters often include water or lubricant in packages thereof for lubrication the intermittent catheters prior to insertion. A user often dumps most of the water or lubricant out of a package before catheterization to mitigate any mess via spillage of the water of lubricant. What is needed is a simple-to-use intermittent catheter that facilitates cleanliness before and after use.

Disclosed herein are intermittent-catheter assemblies and methods thereof that address the foregoing.

### SUMMARY

Disclosed herein is an intermittent-catheter assembly including, in some embodiments, an intermittent catheter and a catheter housing. The intermittent catheter includes a proximal piece including a neck, a cap configured to cap a proximal opening of the proximal piece, and a catheter tube fluidly connected to the proximal piece. The catheter housing is around at least the catheter tube. The catheter housing includes a distal piece and a collapsible sheath. The distal piece includes a chamber or receptacle including a lubricant or lubricating sponge for lubricating the catheter tube when the distal piece is proximally slid over the catheter tube. The collapsible sheath includes a distal portion coupled to the distal piece and a proximal portion coupled to the neck of the proximal piece of the intermittent catheter. An entirety of the catheter tube is disposed in the catheter housing in a packaged state of the intermittent-catheter assembly.

In some embodiments, the cap is configured to cap the proximal piece when not actively placing the intermittent catheter for voiding urine, voiding urine, or removing the intermittent catheter after voiding urine.

In some embodiments, the cap is configured to sit over a flange in a proximal portion of the proximal piece. The flange includes a gasket over the flange configured to sit between the cap and the proximal piece and form a fluidly tight seal when the cap caps the proximal piece.

In some embodiments, the cap is coupled to the proximal piece by a living hinge.

In some embodiments, a proximal portion of the cap includes a receptacle configured to ouroborosly accept a ribbed portion of the distal piece of the catheter housing inserted therein.

In some embodiments, the distal piece inserted into the receptacle of the cap forms an after-use storage state of the intermittent-catheter assembly configured to prevent urine leakage from the intermittent-catheter assembly after voiding urine therewith.

In some embodiments, the catheter tube includes a plurality of eyelets proximate a catheter tip. The eyelets are fluid communication with the proximal opening of the proximal piece.

In some embodiments, the intermittent-catheter assembly further includes a removable foil seal over a distal opening of the distal piece in the packaged state of the intermittent-catheter assembly. The foil seal is configured to maintain sterility of the intermittent catheter prior to use thereof.

In some embodiments, the foil seal is further configured to retain the lubricant in the intermittent-catheter assembly while the intermittent-catheter assembly is in the packaged state thereof.

In some embodiments, the catheter housing further includes an internal piece slidably disposed around the catheter tube. The internal piece is coupled to the sheath between a proximal portion of the sheath and a distal portion of the sheath providing support to the sheath.

In some embodiments, the proximal portion of the sheath is pleated or bellowed and the distal portion of the sheath is not pleated or bellowed.

In some embodiments, the distal piece and the internal piece are configured for placing the intermittent catheter. The distal piece and the internal piece form a two-piece handle for placing the intermittent catheter when at least the distal piece is proximally slid over the catheter tube to the internal piece, which sliding also exposes a distal portion of the catheter tube for placing the intermittent catheter.

Also disclosed herein is a package of intermittent-catheter assemblies including, in some embodiments, resealable packaging and a set of intermittent-catheter assemblies disposed therein. The packaging includes a kerfed portion, a removable piece, and a main body. The kerfed portion includes kerfs configured for tearing the removable piece off the package. The main body includes a cavity and a ziplock in an end portion of the main body for resealing the package after removing the removable piece. The set of intermittent-catheter assemblies are disposed in the cavity of the packaging. Each intermittent-catheter assembly of the one-or-more intermittent-catheter assemblies is in a packaged state thereof.

Also disclosed herein is a carrying case for one or more intermittent-catheter assemblies including, in some embodiments, a main body, a top, and a cavity formed between the main body and the top. The top of the carrying case is coupled to the main body of the carrying case by a living hinge. The cavity, which is formed between the main body and the top, is configured to contain the one-or-more intermittent-catheter assemblies in one or more states thereof.

Also disclosed herein is a method of an intermittent-catheter assembly including, in some embodiments, a catheter obtaining step, an uncapping step, a catheter tube-exposing step, a catheter tube-inserting step, and a urine-voiding step. The catheter obtaining step includes obtaining the intermittent-catheter assembly in a packaged state thereof. The intermittent-catheter assembly includes a catheter housing including a sheath around at least a catheter tube of an intermittent catheter. The uncapping step includes uncapping a cap capping a proximal opening of a proximal piece of the intermittent catheter. The cap is coupled to the proximal piece by a living hinge. The catheter tube-exposing step includes exposing an insertable portion of the catheter tube, which is effectuated by proximally sliding a distal piece of the catheter housing over the catheter tube, which, in turn, lubricates the catheter tube with a lubricant or lubricating sponge in a chamber or receptacle of the distal piece. The catheter tube-inserting step includes inserting the catheter tube into a urethra. The urine-voiding step includes voiding urine from a bladder upon proper placement of the intermittent catheter therein.

In some embodiments, the intermittent-catheter assembly in the packaged state thereof is further packaged in a package of intermittent-catheter assemblies providing about a day's supply thereof.

In some embodiments, the method further includes a foil-removing step. The foil-removing step includes removing a foil seal from a distal opening of the distal piece before the catheter tube-exposing step.

In some embodiments, proximally sliding the distal piece over the catheter tube in accordance with the catheter tube-exposing step includes sliding the distal piece to an internal piece of the catheter housing to form a two-piece handle for inserting the catheter tube into the urethra. A distal portion of the sheath collapses between the distal piece and the internal piece when the distal piece is slid to the internal piece.

In some embodiments, the catheter tube-exposing step further includes proximally sliding the internal piece of the catheter housing or the two-piece handle over the catheter tube to the proximal piece of the intermittent catheter. A proximal portion of the sheath collapses between the internal piece and the proximal piece when the internal piece or the two-piece handle is slide to the proximal piece.

In some embodiments, the method further includes a catheter tube-removing step, a capping step, and a catheter tube-covering step. The catheter tube-removing step includes removing the catheter tube from the urethra after the urine-voiding step. The capping step includes capping the proximal opening of the proximal piece with the cap. The catheter tube-covering step includes covering the insertable portion of the catheter tube by distally sliding the distal piece over the catheter tube.

In some embodiments, the method further includes a joining step. The joining step includes inserting a ribbed portion of the distal piece into a receptacle in a proximal portion of the cap by bending proximal and distal ends of the intermittent-catheter assembly toward each other. The joining step forms an after-use storage state of the intermittent-catheter assembly configured to prevent urine leakage from the intermittent-catheter assembly after the urine-voiding step.

In some embodiments, the method further includes a catheter assembly-storing step. The catheter assembly-storing step includes placing the intermittent-catheter assembly in the after-use storage state thereof into a multiple-use hard-shell carrying case for future disposal of the intermittent-catheter assembly.

Also disclosed herein is another intermittent-catheter assembly including, in some embodiments, an intermittent catheter and a catheter housing coupled to the intermittent catheter. The intermittent catheter includes a proximal piece and a catheter tube fluidly connected to the proximal piece. The proximal piece includes a neck. The catheter housing includes a distal piece and a collapsible sheath. The distal piece is configured to proximally slide over the catheter tube. The collapsible sheath includes a distal portion coupled to the distal piece and a proximal portion coupled to the neck of the proximal piece of the intermittent catheter. An entirety of the catheter tube is disposed in the catheter housing in a packaged state of the intermittent-catheter assembly.

In some embodiments, a distal portion of the distal piece includes a flared receptacle and a proximal portion of the proximal piece includes a flange. The receptacle is configured to ouroborosly accept insertion of the flange therein.

In some embodiments, each piece of the distal piece and the proximal piece includes a medial portion having circumferential ridges. The circumferential ridges are configured for gripping the distal and proximal pieces when inserting the flange into the receptacle.

In some embodiments, the circumferential ridges of the distal piece are also configured for gripping the distal piece when sliding the distal piece over the catheter tube toward the proximal piece to unsheath the catheter tube. The circumferential ridges of the distal piece are also configured for gripping the distal piece when sliding the distal piece over the catheter tube away from the proximal piece to resheath the catheter tube.

In some embodiments, the receptacle includes an inner lip and the flange includes an integrated gasket. The inner lip and the gasket are configured to form a fluidly tight seal when the flange is inserted into the receptacle.

In some embodiments, the flange inserted into the receptacle forms the packaged state of the intermittent-catheter assembly. The packaged state of the intermittent-catheter assembly is configured to retain a hydrophilic coating over the catheter tube, retain a moisture content of the hydrophilic coating, and maintain sterility of the intermittent catheter prior to use thereof.

In some embodiments, the flange inserted into the receptacle after use of the intermittent catheter forms an after-use storage state of the intermittent-catheter assembly. The after-use storage state of the intermittent-catheter assembly is configured to prevent urine leakage from the intermittent-catheter assembly of any residual urine present in the intermittent catheter after voiding urine therewith.

In some embodiments, the catheter tube includes a plurality of eyelets proximate a catheter tip, the eyelets in fluid communication with a proximal opening of the proximal piece.

Also disclosed herein is another method of an intermittent-catheter assembly including, in some embodiments, a catheter-obtaining step, a flange-removing step, a catheter tube-exposing step, a catheter tube-inserting step, and a urine-voiding step. The catheter-obtaining step includes obtaining the intermittent-catheter assembly in a packaged state thereof. The intermittent-catheter assembly includes a catheter housing including a sheath around at least a catheter tube of an intermittent catheter. The flange-removing step includes removing a flange in a proximal portion of a proximal piece of the intermittent catheter from a flared receptacle of a distal portion of a distal piece of the catheter housing. The catheter tube-exposing step includes exposing an insertable portion of the catheter tube including proximally sliding the distal piece over the catheter tube toward the proximal piece. The sheath collapses between the distal piece and the proximal piece with the sliding of the distal piece over the catheter tube, thereby unsheathing the catheter tube. The catheter tube-inserting step includes inserting the catheter tube into a urethra. The urine-voiding step includes voiding urine from a bladder upon proper placement of the intermittent catheter therein.

In some embodiments, the flange is ouroborosly inserted in the receptacle in packaged state of the intermittent-catheter assembly.

In some embodiments, proximally sliding the distal piece over the catheter tube toward the proximal piece forms a two-piece handle between the distal and proximal pieces for the catheter tube-inserting step.

In some embodiments, the method further includes a catheter tube-removing step, a catheter tube-resheathing step, a catheter assembly-bending step, and a joining step. The catheter tube-removing step includes removing the catheter tube from the urethra after the voiding of the urine from the bladder. The catheter tube-resheathing step includes covering the insertable portion of the catheter tube with the sheath by distally sliding the distal piece over the catheter tube, thereby resheathing the catheter tube. The catheter assembly-bending step includes bending proximal and distal portions of the intermittent-catheter assembly toward each other. The joining step includes ouroborosly inserting the flange of the proximal piece into the receptacle of the distal piece, thereby forming an after-use storage state of the intermittent-catheter assembly. The after-use storage state of the intermittent-catheter assembly is configured to prevent urine leakage from the intermittent-catheter assembly after the voiding of the urine from the bladder.

In some embodiments, the method further includes a storing step. The storing step includes placing the intermittent-catheter assembly in the after-use storage state thereof into a multiple-use hard-shell carrying case for future disposal of the intermittent-catheter assembly.

In some embodiments, the intermittent-catheter assembly in the packaged or after-use storage state thereof is further packaged in the carrying case with about a day's supply of other intermittent-catheter assemblies.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a perspective view of an intermittent-catheter assembly in accordance with some embodiments.

FIG. 2 illustrates a side view of the intermittent-catheter assembly of FIG. 1 in a packaged state thereof in accordance with some embodiments.

FIG. 3 illustrates a side view of the intermittent-catheter assembly of FIG. 1 when uncapped in accordance with some embodiments.

FIG. 4 illustrates a side view of the intermittent-catheter assembly of FIG. 1 with a fully exposed catheter tube in accordance with some embodiments.

FIG. 5 illustrates a side view of the intermittent-catheter assembly of FIG. 1 when capped in accordance with some embodiments.

FIG. 6A illustrates a longitudinal cross-section of a distal portion of the intermittent-catheter assembly of FIG. 1 including a lubricant-filled chamber in accordance with some embodiments.

FIG. 6B illustrates the longitudinal cross-section of the distal portion of the intermittent-catheter assembly of FIG. 1 including a lubricating sponge-filled chamber in accordance with some embodiments.

FIG. 6C illustrates the longitudinal cross-section of the distal portion of the intermittent-catheter assembly of FIG. 1 including a lubricating sponge-filled receptacle in accordance with some embodiments.

FIG. 7 illustrates a perspective view of a proximal portion of the intermittent-catheter assembly of FIG. 1 when capped in accordance with some embodiments.

FIG. 8 illustrates a perspective view of the proximal portion of the intermittent-catheter assembly of FIG. 1 when uncapped in accordance with some embodiments.

FIG. 9 illustrates a detailed view of joining a distal piece of a catheter housing of the intermittent-catheter assembly of FIG. 1 to a cap of an intermittent catheter of the intermittent-catheter assembly in accordance with some embodiments.

FIG. 10 illustrates a perspective view of the distal piece inserted into the cap of the intermittent catheter forming an after-use storage state of the intermittent-catheter assembly of FIG. 1 in accordance with some embodiments.

FIG. 11 illustrates a package of intermittent-catheter assemblies such as the intermittent catheter assembly of FIG. 1 in accordance with some embodiments.

FIG. 12 illustrates a carrying case for one or more intermittent-catheter assemblies such as the intermittent catheter assembly of FIG. 1 in accordance with some embodiments.

FIG. 13 illustrates a side view of another intermittent-catheter assembly in accordance with some embodiments.

FIG. 14 illustrates a perspective view of a distal portion of the intermittent-catheter assembly of FIG. 13 in accordance with some embodiments.

FIG. 15 illustrates a perspective view of a proximal portion of the intermittent-catheter assembly of FIG. 13 in accordance with some embodiments.

FIG. 16 illustrates a perspective view of a distal piece of the catheter housing inserted into a proximal piece of the intermittent catheter forming a packaged state or an after-use storage state of the intermittent-catheter assembly of FIG. 13 in accordance with some embodiments.

FIG. 17 illustrates a detailed view of the distal piece inserted into the proximal piece of the intermittent catheter forming the packaged state or the after-use storage state of the intermittent-catheter assembly of FIG. 13 in accordance with some embodiments.

FIG. 18 illustrates a carrying case for one or more intermittent-catheter assemblies such as the intermittent catheter assembly of FIG. 13 in accordance with some embodiments.

FIG. 19 illustrates removal of the intermittent catheter assembly of FIG. 13 from the carrying case of FIG. 18 or disposal of the intermittent catheter assembly in the carrying case in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, catheterization with intermittent catheters can be messy. Indeed, existing intermittent catheters often include water or lubricant in packages thereof for lubrication the intermittent catheters prior to insertion. A user often dumps most of the water or lubricant out of a package before catheterization to mitigate any mess via spillage of the water of lubricant. What is needed is a simple-to-use intermittent catheter that facilitates cleanliness before and after use.

Disclosed herein are intermittent-catheter assemblies and methods thereof that address the foregoing.

### Intermittent-Catheter Assemblies

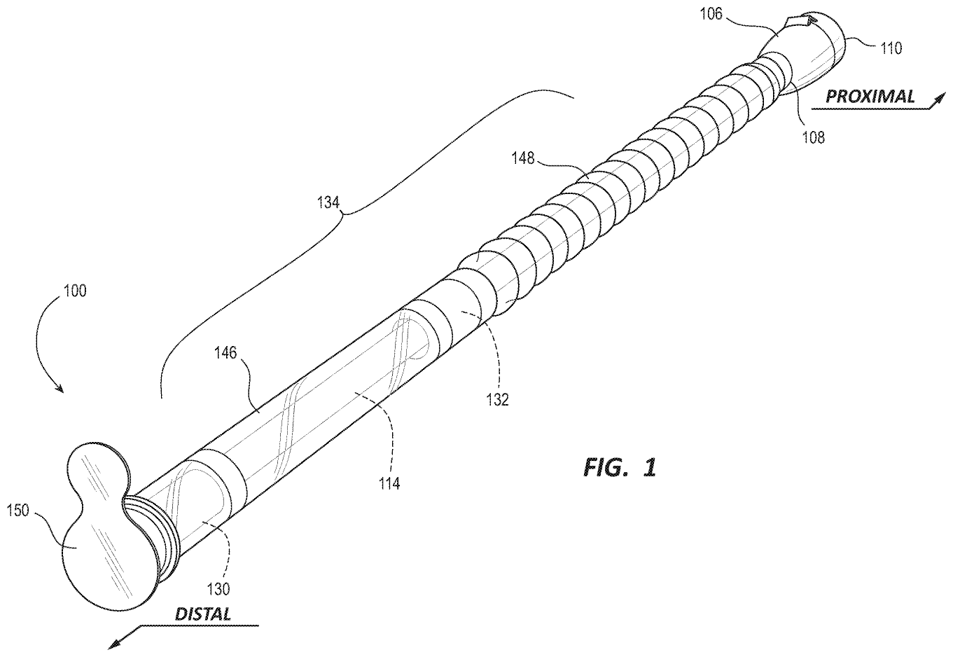

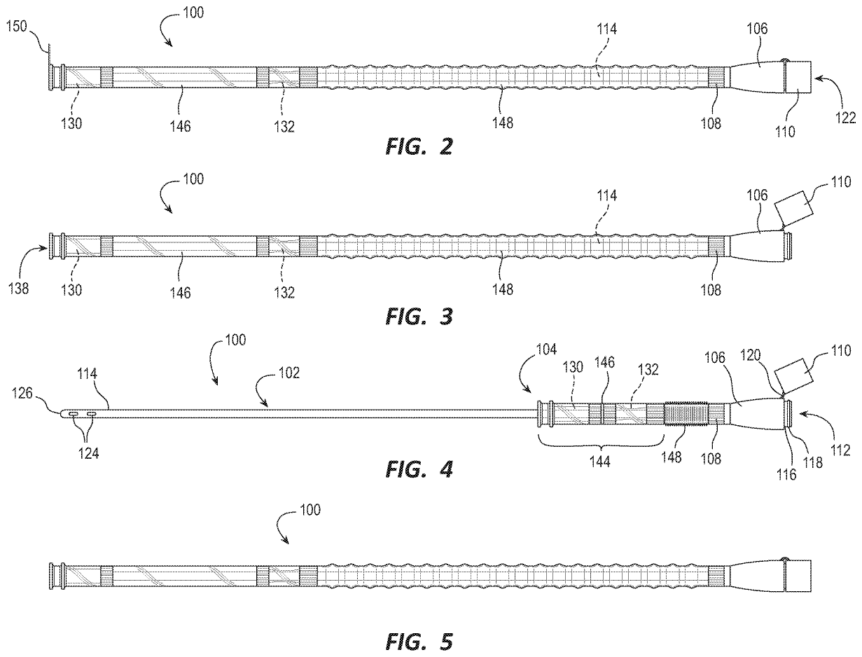

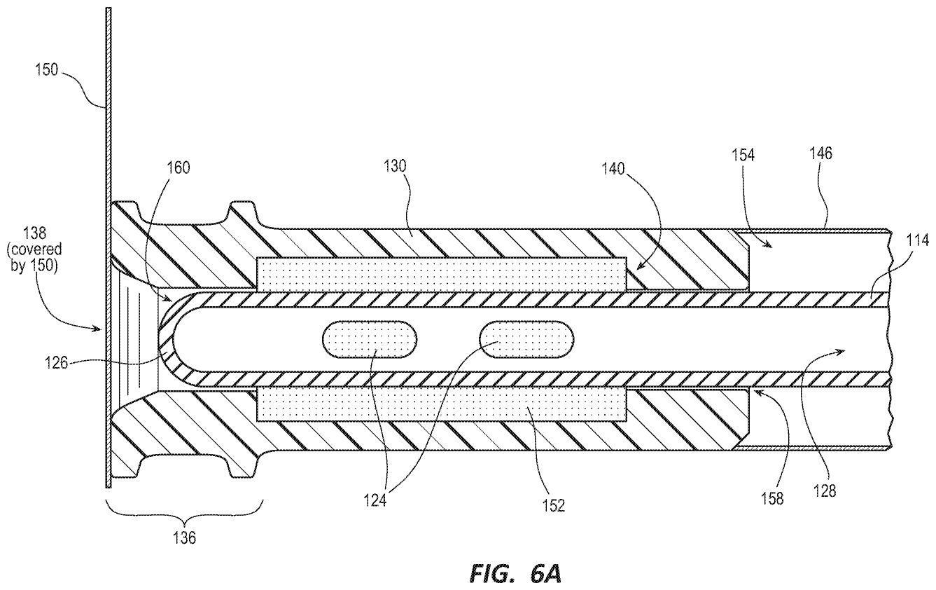

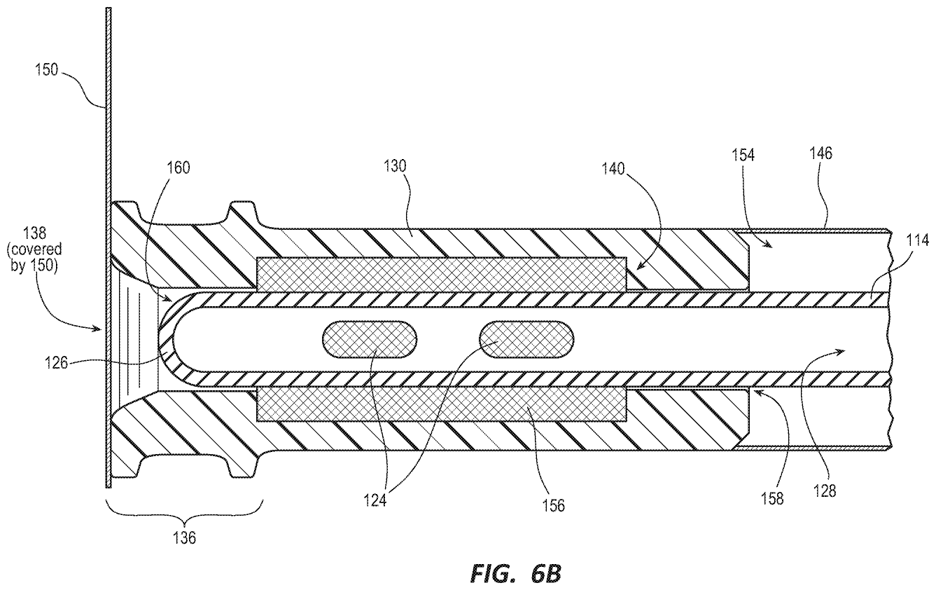

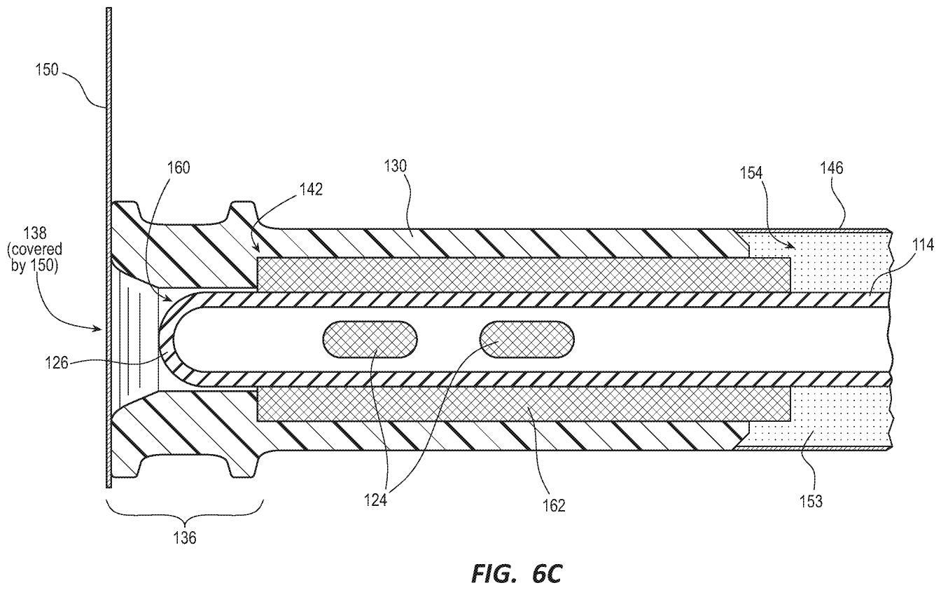

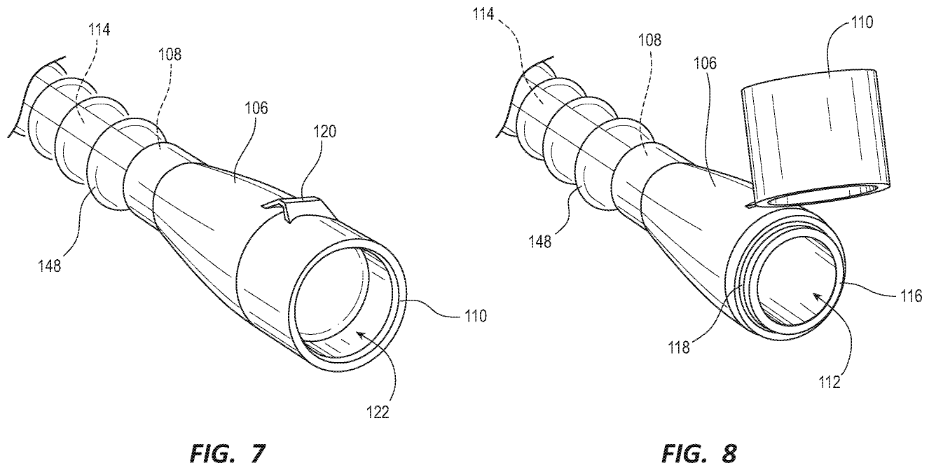

FIGS. 1-5 illustrate various views of an intermittent-catheter assembly 100 in various states accordance with some embodiments. For example: The intermittent-catheter assembly 100 of FIGS. 1 and 2 are in a packaged state thereof as set forth below. The intermittent-catheter assembly 100 of FIG. 3 is in a pre-insertion state thereof such as before the catheter tube-exposing step of the method set forth below. The intermittent-catheter assembly 100 of FIG. 4 is in a pre-insertion state thereof such as after the catheter tube-exposing step of the method set forth below, an insertion state thereof such as during the urine-voiding step of the method set forth below, or in a post-insertion state thereof such as after the catheter tube-removing step of the method set forth below. The intermittent-catheter assembly 100 of FIG. 5 is in a pre-joined state thereof such as before the joining step of the method set forth below. Continuing with FIGS. 6A-6C, 7, and 8, a longitudinal cross-section of a distal portion of the intermittent-catheter assembly 100 is illustrated in FIGS. 6A-6C in accordance with some embodiments, whereas FIGS. 7 and 8 illustrate a perspective view of a proximal portion of the intermittent-catheter assembly 100 in accordance with some embodiments.

As shown, the intermittent-catheter assembly 100 includes, in some embodiments, an intermittent catheter 102 (e.g., a male intermittent catheter) and a catheter housing 104. Description for each of the primary components of the intermittent-catheter assembly 100 (i.e., the intermittent catheter 102 and the catheter housing 104) is set forth, in turn, below. Following the description for the primary components of the intermittent-catheter assembly 100 is description for additional features of the intermittent-catheter assembly 100.

The intermittent catheter 102 includes a proximal piece 106 including a neck 108, a cap 110 configured to cap a proximal opening 112 of the proximal piece 106, and a catheter tube 114 fluidly connected to the proximal piece 106.

The proximal piece 106 is configured to provide a handle for holding the intermittent-catheter assembly 100 while voiding urine through the proximal opening 112 of the proximal piece 106. The proximal piece 106 includes a flange 116 with a gasket 118 (e.g., ‘O’-ring) thereover in a proximal portion of the proximal piece 106. The gasket 118 is configured to sit between the flange 116 and the cap 110 and form a fluidly tight seal when the cap 110 caps the proximal piece 106.

The cap 110 (e.g., a flip-top cap) is configured to cap the proximal piece 106 when not actively placing the intermittent catheter 102 for voiding urine, voiding urine, or removing the intermittent catheter 102 after voiding urine. The cap 110 is coupled to the proximal piece 106 by a living hinge 120. The cap 110 is configured to sit over the flange 116 of the proximal piece 106 such that the gasket 118 is between the cap 110 and the flange 116, thereby forming a fluidly tight seal when the cap 110 caps the proximal piece 106.

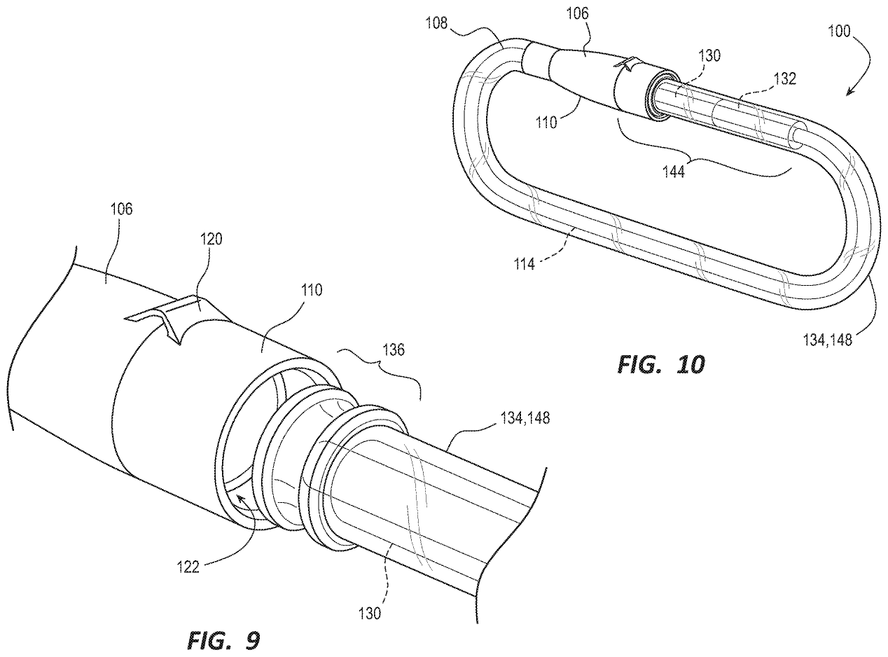

The cap 110 is also configured for effectuating an after-use storage state of the intermittent-catheter assembly 100 as set forth below. Indeed, the cap 110 includes a receptacle 122 for the ribbed portion 136 of the distal piece 130 of the catheter housing 104 set forth below for effectuating the after-use storage state of the intermittent-catheter assembly 100. The after-use storage state of the intermittent-catheter assembly 100 prevents urine leakage from the intermittent-catheter assembly 100 after voiding urine therewith. (See, for example, FIGS. 9 and 10.)

The catheter tube 114 is configured for insertion into a urethra for voiding urine from a bladder. The catheter tube 114 in one embodiment includes a plurality of eyelets 124 proximate a catheter tip 126. The eyelets 124 are fluid communication with the proximal opening 112 of the proximal piece 106 by way of a catheter-tube lumen 128 extending along a length of the catheter tube 114. Although two eyelets 124 are shown, any number or shape of openings are possible and within the scope of the invention, such as one large opening, more than two openings, etc. Also, the one-or-more openings can be spaced around the circumference of the catheter tube 114.

The catheter housing 104 includes a distal piece 130, an internal piece 132, and a sheath 134. An entirety of the catheter tube 114 is disposed in the catheter housing 104 in the packaged state of the intermittent-catheter assembly 100 as shown in FIGS. 1 and 2. However, the catheter housing 104 is around at least a portion of the catheter tube 114 in an any state of the intermittent-catheter assembly 100. (See, for example, FIGS. 1 and 2, 4, and 10.)

The distal piece 130 is configured to form the two-piece handle 144 with the internal piece 132 as set forth below for placing the intermittent catheter 102. The distal piece 130 is thusly slidably disposed around the catheter tube 114 for sliding the distal piece 130 to the internal piece 132.

The distal piece 130 is also configured for effectuating the after-use storage state of the intermittent-catheter assembly 100 as set forth below. Indeed, the distal piece 130 includes a ribbed portion 136 in a distal portion of the distal piece 130 for effectuating the after-use storage state of the intermittent-catheter assembly 100. Again, the after-use storage state of the intermittent-catheter assembly 100 prevents urine leakage from the intermittent-catheter assembly 100 after voiding urine therewith. (See, for example, FIGS. 9 and 10.)

The distal piece 130 is also configured for mitigating liquid (e.g., water, lubricant, or a combination thereof) migration into the eyelets 124 of the catheter tube 114, through the lumen 128 of the catheter tube 114, and out the proximal opening 112 of the proximal piece 106 when uncapped or out of a distal opening 138 of the distal piece 130 when the foil seal 150 is removed, each of which mitigates any mess that might otherwise occur when a user commences using the intermittent-catheter assembly 100. Indeed, the distal piece 130 includes a chamber 140 or a receptacle 142 for mitigating the liquid migration as set forth below.

The internal piece 132 is configured to form a two-piece handle 144 with the distal piece 130 for at least placing the intermittent catheter 102. The internal piece 132 is thusly slidably disposed around the catheter tube 114 for independently sliding the internal piece 132 or sliding the internal piece 132 together with the distal piece 130. Indeed, the distal piece 130 and the internal piece 132 form the two-piece handle 144 for placing the intermittent catheter 102 when at least the distal piece 130 is proximally slid over the catheter tube 114 to the internal piece 132, which, incidentally, also exposes a distal portion of the catheter tube 114 for placing the intermittent catheter 102, or the internal piece 132 is distally slid over the catheter tube 114 to the distal piece 130. FIG. 4 illustrates the former in which the two-piece handle 144 is in a proximal position of the intermittent-catheter assembly 100 for the pre-insertion state thereof such as after the catheter tube-exposing step of the method set forth below, the insertion state thereof such as during the urine-voiding step of the method set forth below, or in the post-insertion state thereof such as after the catheter tube-removing step of the method set forth below. FIG. 10 illustrates the latter in which the two-piece handle 144 is in a distal position of the intermittent-catheter assembly 100 for the after-use storage state thereof.

The internal piece 132 is also configured to provide an internal structural support for the sheath 134. Indeed, the internal piece 132 is coupled to the sheath 134 between the distal portion 146 of the sheath 134 and the proximal portion 148 of the sheath 134 set forth below providing structural support to the sheath 134.

The sheath 134 is configured to maintain sterility of the catheter tube 114 in at least the packaged state of the intermittent-catheter assembly 100, as well as retain urine in the after-use storage state of the intermittent-catheter assembly 100.

The sheath 134 is collapsible for transitioning between different states of the intermittent-catheter assembly 100. The sheath 134 includes a distal portion 146 coupled to the distal piece 130, a proximal portion 148 coupled to the neck 108 of the proximal piece 106, a portion between the distal portion 146 and the proximal portion 148 of the sheath 134 (e.g., a medial portion of the sheath 134) coupled to the internal piece 132. The proximal portion 148 of the sheath 134 is pleated or bellowed for extension over the catheter tube 114 in the after-use storage state of the intermittent-catheter assembly 100. (See FIG. 10.) However, the distal portion 146 of the sheath 134 need not pleated or bellowed as shown in the illustrated embodiment.

The intermittent-catheter assembly 100 further includes a removable foil seal 150 over the distal opening 138 of the distal piece 130 of the catheter housing 104 in the packaged state of the intermittent-catheter assembly 100. (See FIGS. 1 and 2.) The foil seal 150 is configured to maintain sterility of the intermittent catheter 102 prior to use thereof.

The intermittent-catheter assembly 100 can further include a liquid (e.g., a lubricant 152, water 153, or a combination thereof) disposed in the intermittent-catheter assembly 100 in a sheath lumen 154 between the catheter tube 114 and the sheath 134 for lubricating the catheter tube 114. The foil seal 150 is further configured to retain the liquid in the intermittent-catheter assembly 100 while the intermittent-catheter assembly 100 is in the packaged state thereof. Additional or alternative lubricating means for lubricating the catheter tube 114 are respectively shown in FIGS. 6A-6C, each of which includes a liquid-retaining means for retaining the liquid (e.g., the lubricant 152, the water 153, or the combination thereof) in the intermittent-catheter assembly 100 as well.

FIGS. 6A-6C illustrate the longitudinal cross-section of the distal portion of the intermittent-catheter assembly 100 respectively including a lubricant-filled chamber 140 in the distal piece 130, a lubricating sponge-filled chamber 140 in the distal piece 130, and a lubricating sponge-filled receptacle 142 in the distal piece 130 in accordance with some embodiments.

The lubricating means shown in FIGS. 6A-6C is configured for lubricating the catheter tube 114 as it passes through the chamber 140 or the receptacle 142 (e.g., as the distal piece 130 is proximally slid over the catheter tube 114). The associated liquid-retaining means is configured for mitigating the liquid (e.g., the lubricant 152, the water 153, or the combination thereof) from migrating into the eyelets 124 of the catheter tube 114, through the lumen 128 of the catheter tube 114, and out of the proximal opening 112 of the proximal piece 106 when uncapped or migrating out of the distal opening 138 of the distal piece 130 when the foil seal 150 is removed. Mitigating the liquid from migrating out of the proximal opening 112 of the proximal piece 106 or the distal opening 138 of the distal piece 130 mitigates any mess that might otherwise occur when a user commences using the intermittent-catheter assembly 100 (e.g., uncapping the cap 110 capping the proximal opening 112 of the proximal piece 106 of the intermittent catheter 102, removing the foil seal 150 from the distal opening 138 of the distal piece 130, etc.).

As shown, the distal piece 130 of the intermittent-catheter assembly 100 in each figure of FIGS. 6A and 6B includes the chamber 140, wherein the chamber 140 shown in FIG. 6A includes the liquid (e.g., the lubricant 152, the water 153, or the combination thereof) for lubricating the catheter tube 114 as needed, and wherein the chamber 140 shown in FIG. 6B includes a lubricating sponge 156 (e.g., a sponge impregnated with the liquid) for lubricating the catheter tube 114 as needed. Due to a narrow cylindrical gap 158 between the chamber 140 and the sheath lumen 154 proximal of the distal piece 130, ingress of any of the liquid into the chamber 140 from the sheath lumen 154 is minimal to none, particularly if the liquid is relatively viscous (e.g., a lubricating gel). This limits an amount of the liquid that can migrate into the eyelets 124 of the catheter tube 114 and out the proximal opening 112 of the proximal piece 106 when uncapped. Similarly, a narrow cylindrical gap 160 between the chamber 140 and the distal opening 138 of the distal piece 130 limits egress of any of the liquid out of the chamber 140, thereby limiting an amount of the liquid that can migrate out of the distal opening 138 of the distal piece 130 when the foil seal 150 is removed. As to the lubricating sponge 156, the lubricating sponge 156 further mitigates ingress or egress of any of the liquid into or out of the chamber 140 on account of its rate-limiting wicking mechanism. Furthermore, the lubricating sponge 156 does not wick any of the liquid into the chamber 140 from the sheath lumen 154 when the lubricating sponge is already saturated.

While the distal piece 130 of the intermittent-catheter assembly 100 in FIG. 6C includes the receptacle 142 instead of the chamber 140 of the intermittent-catheter assembly 100 shown in FIG. 6A or 6B, the receptacle 142 still limits egress of any of the liquid (e.g., the lubricant 152, the water 153, or the combination thereof) out of the receptacle 142 through the narrow cylindrical gap 160 between the receptacle 142 and the distal opening 138 of the distal piece 130 like the chamber 140 set forth above. In addition, a lubricating sponge 162 having a distal portion disposed in the liquid in the sheath lumen 154 also mitigates ingress or egress of any of the liquid into or out of the receptacle 142 on account of its rate-limiting wicking mechanism and reluctance to wick any of the liquid into the receptacle 142 from the sheath lumen 154 when the lubricating sponge is already saturated therewith.

FIG. 9 illustrates a detailed view of joining the distal piece 130 of the catheter housing 104 to the cap 110 of the intermittent catheter 102 in accordance with some embodiments. FIG. 10 illustrates a perspective view of the distal piece 130 of the catheter housing 104 inserted into the cap 110 of the intermittent catheter 102 forming the after-use storage state of the intermittent-catheter assembly 100 in accordance with some embodiments.

As alluded to above, the cap 110 and the distal piece 130 are both configured to effectuate the after-use storage state of the intermittent-catheter assembly 100. Indeed, the cap 110 includes the receptacle 122 and the distal piece 130 includes the ribbed portion 136, which receptacle 122 is configured to accept the ribbed portion 136 inserted therein when the intermittent-catheter assembly 100 is bent in an end-to-end fashion akin to that of a dragon or serpent eating its own tail (e.g., the ouroboros of ancient Egyptian iconography). The distal piece 130 is further configured to effectuate the after-use storage state of the intermittent-catheter assembly 100 together with the internal piece 132 of the catheter housing 104 in the two-piece handle 144. Indeed, when the two-piece handle 144 is in the distal position of the intermittent-catheter assembly 100 in the after-use storage state thereof as shown in FIG. 10, the two-piece handle 144 provides structural support to counter circumferential stress in that location while the intermittent-catheter assembly 100 is in the after-use storage state thereof. Again, the after-use storage state of the intermittent-catheter assembly 100 prevents urine leakage from the intermittent-catheter assembly 100 after voiding urine therewith.

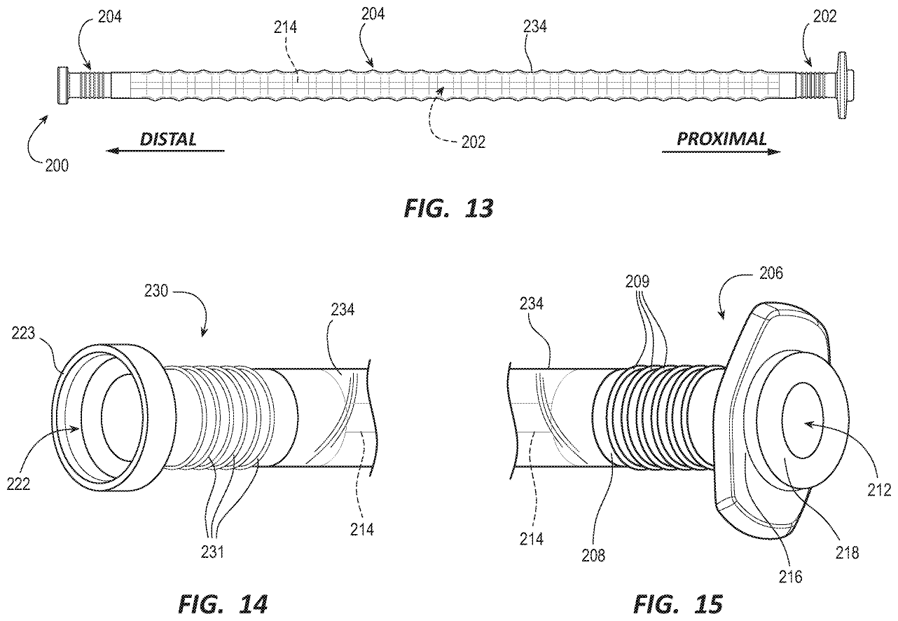

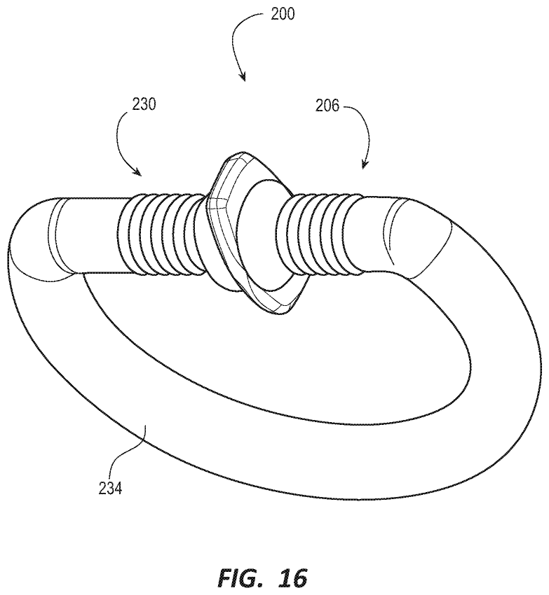

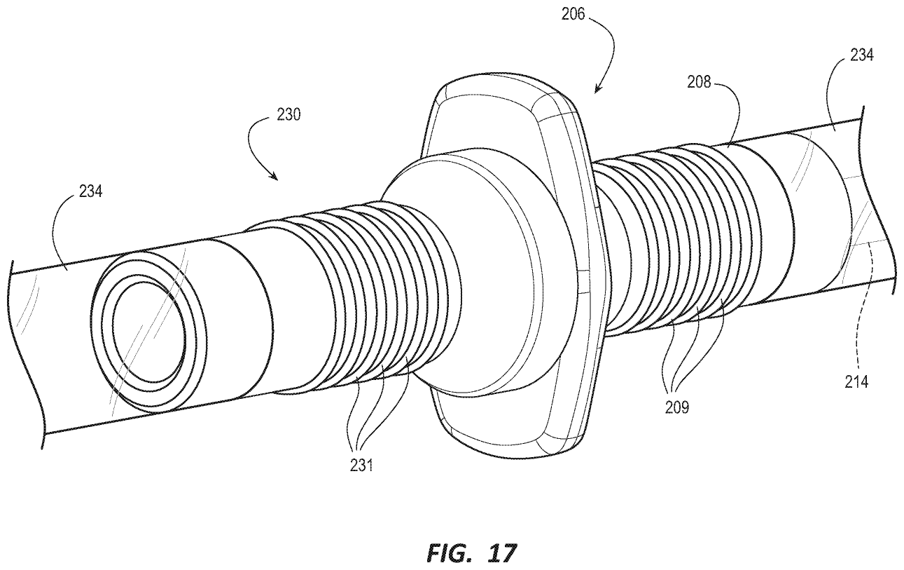

FIGS. 13-17 illustrate various views of an intermittent-catheter assembly 200 in various states accordance with some embodiments. For example: The intermittent-catheter assembly 200 of FIG. 13 is in a pre-insertion state thereof such as before the catheter tube-exposing step of the method set forth below. Alternatively, the intermittent-catheter assembly 200 of FIG. 13 is in a pre-joined state thereof such as before the joining step of the method set forth below. The intermittent-catheter assembly 200 of FIG. 16 is in a packaged state thereof as set forth below. Alternatively, the intermittent-catheter assembly 200 of FIG. 16 is in an after-use storage state thereof as set forth below.

As shown, the intermittent-catheter assembly 200 includes, in some embodiments, an intermittent catheter 202 (e.g., a male intermittent catheter) and a catheter housing 204 coupled to the intermittent catheter 202. Description for each of the primary components of the intermittent-catheter assembly 200 (i.e., the intermittent catheter 202 and the catheter housing 204) is set forth, in turn, below.

The intermittent catheter 202 includes a proximal piece 206 including a neck 208 as well as a catheter tube 214 fluidly connected to the proximal piece 206.

The proximal piece 206 is configured to provide a handle or a portion of the two-piece handle set forth below for holding the intermittent-catheter assembly 200 while voiding urine through the proximal opening 212 of the proximal piece 206. Indeed, the proximal piece 206 includes a medial portion having circumferential ridges 209 configured for gripping the proximal piece 206 while voiding urine through the proximal opening 212 of the proximal piece 206. The proximal piece 206 includes a flange 216 with an integrated gasket 218 (e.g., an integrated ‘O’-ring) thereover in a proximal portion of the proximal piece 206. The gasket 218 is configured to form a fluidly tight seal when the flange 216 is inserted into the receptacle 222 set forth below. Notably, the circumferential ridges 209 are also configured for gripping the proximal piece 206 when inserting the flange 216 into the receptacle 222.

The catheter tube 214 is configured for insertion into a urethra for voiding urine from a bladder. As to insertion into the urethra, the catheter tube 214 includes an activated or wetted hydrophilic coating over the catheter tube 214. As to voiding urine from the bladder, the catheter tube 214 in one embodiment includes a plurality of eyelets proximate a catheter tip. (See, for example, the eyelets 124 and the catheter tip 126 of the catheter tube 114 in FIG. 4.) The eyelets are fluid communication with the proximal opening 212 of the proximal piece 206 by way of a catheter-tube lumen extending along a length of the catheter tube 214. (See, for example, the catheter-tube lumen 128 of the catheter tube 114 in FIGS. 6A-6C.)

The catheter housing 204 includes a distal piece 230 and a sheath 234. An entirety of the catheter tube 214 is disposed in the catheter housing 204 in the packaged state of the intermittent-catheter assembly 200 as shown in FIGS. 16 and 19. However, the catheter housing 204 is around at least a portion of the catheter tube 214 in an any state of the intermittent-catheter assembly 200.

The distal piece 230 is slidably disposed around the catheter tube 214 for proximally sliding the distal piece 230 over the catheter tube 214 to the proximal piece 206 to unsheath the catheter tube 214 for catheterization therewith. The distal piece 230 is slidably disposed around the catheter tube 214 for distally sliding the distal piece 230 over the catheter tube 214 away from the proximal piece 206 to resheath the catheter tube 214 after catheterization therewith. Like the proximal piece 206, the distal piece 230 includes a medial portion having circumferential ridges 231 configured for gripping the distal piece 230 while proximally or distally sliding the distal piece 230 over the catheter tube 214. In addition, the distal piece 230 is configured to form a two-piece handle with the proximal piece 206 when proximally slid over the catheter tube 214 to the proximal piece 206. (See, for example, the proximal piece 106 and the distal piece 130 of the intermittent-catheter assembly 100 in FIG. 4.) Aided by the circumferential ridges 209 and 231 of both the proximal and distal pieces 206 and 230, the two-piece handle is configured for holding the intermittent-catheter assembly 200 while voiding urine through the proximal opening 212 of the proximal piece 206. Notably, the circumferential ridges 231 are also configured for gripping the distal piece 230 when inserting the flange 216 into the receptacle 222 set forth below.

The distal piece 230 is also configured for effectuating both the packaged state and the after-use storage state of the intermittent-catheter assembly 200. Indeed, the distal piece 230 includes a flared receptacle 222 with an inner lip 223 in a distal portion of the distal piece 230 for forming a fluidly tight seal when the flange 216 is inserted into the receptacle 222. Insertion of the flange 216 into the receptacle 222 effectuates both the original, packaged state of the intermittent-catheter assembly 200 as well as the after-use storage state of the intermittent-catheter assembly 200. The after-use storage state of the intermittent-catheter assembly 200 is configured to prevent urine leakage from the intermittent-catheter assembly 200 of any residual urine present in the intermittent catheter 202 after voiding urine therewith. (See, for example, FIGS. 16 and 19.)

The sheath 234 is configured to retain the hydrophilic coating over the catheter tube 214 in the packaged state of the intermittent-catheter assembly 200, maintain a moisture content of the hydrophilic coating in the packaged state of the intermittent-catheter assembly 200, and maintain sterility of the intermittent catheter 202 in the packaged state of the intermittent-catheter assembly 200 prior to use thereof. The sheath 234 is also configured to prevent urine leakage from the intermittent-catheter assembly 200 in the after-use storage state of the intermittent-catheter assembly 200. However, other features of the intermittent-catheter assembly 200 for effectuating the foregoing states of the intermittent-catheter assembly 200 also contribute to the foregoing as well.

The sheath 234 is collapsible for transitioning between different states of the intermittent-catheter assembly 200. The sheath 234 includes a distal portion coupled to the distal piece 230 and a proximal portion coupled to the neck 208 of the proximal piece 206. The sheath 234 is pleated or bellowed to facilitate transitioning between the different states of the intermittent-catheter assembly 200 including the packaged state of the intermittent-catheter assembly 200 and the after-use storage state of the intermittent-catheter assembly 200.

As alluded to above, the distal piece 230 and the proximal piece 206 are both configured to effectuate the packaged state of the intermittent-catheter assembly 200 and the after-use storage state of the intermittent-catheter assembly 200. Indeed, the distal piece 230 includes the receptacle 222 and the proximal piece 206 includes the flange 216, which receptacle 222 is configured to accept the flange 216 inserted therein when the intermittent-catheter assembly 200 is bent in an end-to-end fashion akin to that of a dragon or serpent eating its own tail (e.g., the ouroboros of ancient Egyptian iconography). In the packaged state of the intermittent-catheter assembly 200, the hydrophilic coating over the catheter tube 214 is retained, the moisture content of the hydrophilic coating is maintained, and sterility of the intermittent catheter 202 is maintained prior to use thereof. In the after-use storage state of the intermittent-catheter assembly 200, urine leakage from the intermittent-catheter assembly 200 is prevented.

### Packaged Intermittent-Catheter Assemblies

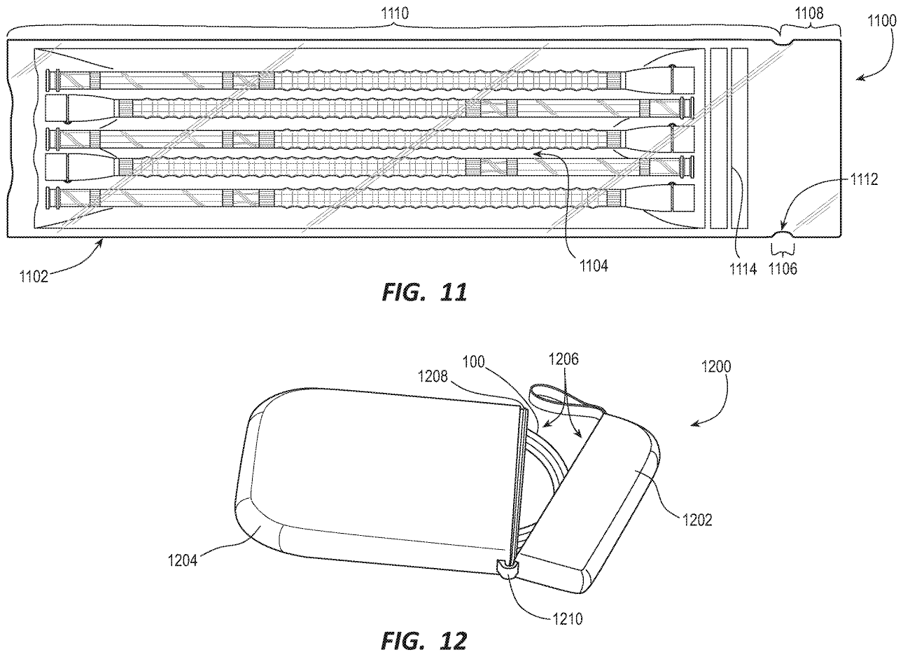

FIG. 11 illustrates a package of intermittent-catheter assemblies 1000 in accordance with some embodiments.

As shown the package of intermittent-catheter assemblies 1000 includes, in some embodiments, resealable packaging 1102 and a set of intermittent-catheter assemblies 1104 disposed therein.

The packaging 1102 includes a kerfed portion 1106, a removable piece 1108, and a main body 1110.

The kerfed portion 1106 of the packaging 1102 includes kerfs 1112 configured for tearing the removable piece 1108 off the package 1000 or the packaging 1102 thereof.

The main body 1110 includes a cavity and a ziplock 1114 in an end portion of the main body 1110 for resealing the package 1000 or the packaging 1102 thereof after removing the removable piece 1108.

The set of intermittent-catheter assemblies 1104 (e.g., about a day's supply) are disposed in the cavity of the packaging 1102. Each intermittent-catheter assembly 100 of the set of intermittent-catheter assemblies 1104 is disposed in the packaging 1102 in its extended form to minimize curvature thereof, which curvature could complicate insertion. IN addition, each intermittent-catheter assembly 100 is disposed in the packaging 1102 in the packaged state thereof as set forth above.

### Carrying Cases

FIG. 12 illustrates a carrying case 1200 for one or more intermittent-catheter assemblies such as the intermittent-catheter assembly 100 in accordance with some embodiments.

As shown, the carrying case 1200 is a multiple-use hard-shell carrying case including, in some embodiments, a top 1202, a main body 1204, and a cavity 1206 formed between the top 1202 and the main body 1204. The carrying case 1200 including the top 1202 and the main body 1204 of the carrying case 1200 can be rigid to prevent damage to any intermittent-catheter assemblies disposed therein and promote longer-term use of the carrying case.

The top 1202 of the carrying case 1200 is configured to sit over a flange 1208 about an open end of the main body 1204 of the carrying case 1200, which facilitates keeping the carrying case 1200 closed by way of an interference fit between the top 1202 and the flange 1208. The top 1202 of the carrying case 1200 is coupled to the main body 1204 of the carrying case 1200 by a living hinge 1210, which facilitates fitting the top 1202 over the flange 1208 of the main body 1204, as well as eliminates misplacement of the top 1202.

The cavity 1206, which is formed between the main body 1204 of the carrying case 1200 and the top 1202 of the carrying case 1200 when the top 1202 is fitted over the open end of the main body 1204, is configured to contain the one-or-more intermittent-catheter assemblies in one or more states thereof.

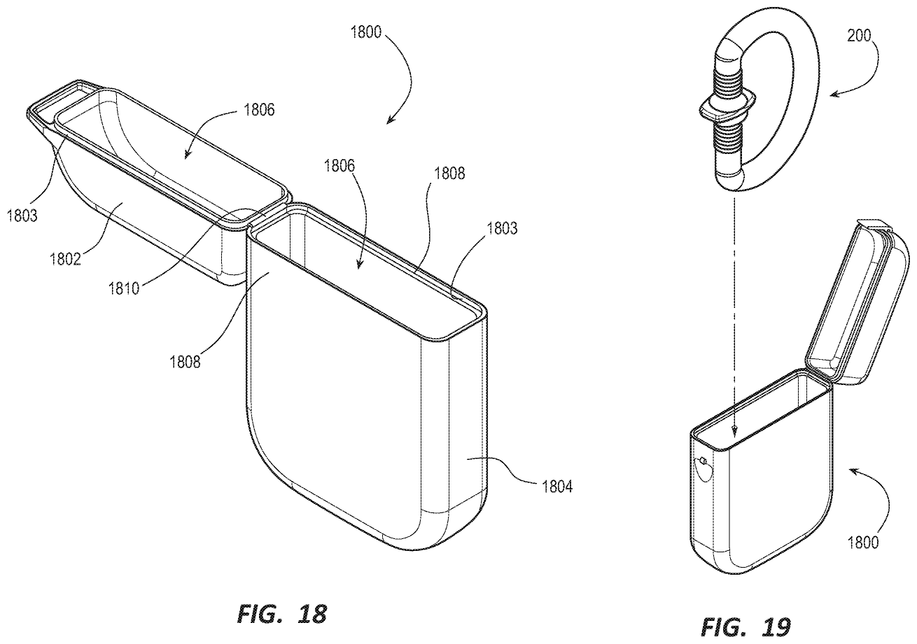

FIG. 18 illustrates a carrying case 1800 for one or more intermittent-catheter assemblies such as the intermittent-catheter assembly 200 in accordance with some embodiments.

Like the carrying case 1200, the carrying case 1800 is a multiple-use hard-shell carrying case including, in some embodiments, a top 1802, a main body 1804, and a cavity 1806 formed between the top 1802 and the main body 1804. The carrying case 1800 including the top 1802 and the main body 1804 of the carrying case 1800 can be rigid to prevent damage to any intermittent-catheter assemblies disposed therein and promote longer-term use of the carrying case 1800.

A perimetrical edge 1803 extending from the top 1802 of the carrying case 1800 is configured to sit on an inner lip 1808 about an open end of the main body 1804 of the carrying case 1800, which facilitates keeping the carrying case 1800 closed by way of an interference fit between the perimetrical edge 1803 and a perimetrical edge 1805 extending from the main body 1804 over the inner lip 1808. The top 1802 of the carrying case 1800 is coupled to the main body 1804 of the carrying case 1800 by a living hinge 1810, which facilitates fitting the top 1802 over the main body 1804, as well as eliminates misplacement of the top 1802.

The cavity 1806, which is formed between the main body 1804 of the carrying case 1800 and the top 1802 of the carrying case 1800 when the top 1802 is fitted over the open end of the main body 1804, is configured to contain the one-or-more intermittent-catheter assemblies in one or more states thereof.

### Methods

Methods of the intermittent-catheter assembly 100 of FIGS. 1-10 include methods of using the intermittent-catheter assembly 100. For example, a method of the intermittent-catheter assembly 100 includes, in some embodiments, a catheter obtaining step, an uncapping step, a catheter tube-exposing step, a catheter tube-inserting step, and a urine-voiding step.

The catheter obtaining step includes obtaining the intermittent-catheter assembly 100 in the packaged state thereof. The intermittent-catheter assembly 100 in the packaged state thereof can be further packaged in the package of intermittent-catheter assemblies 1100 providing about a day's supply thereof. As set forth above, the intermittent-catheter assembly 100 includes the catheter housing 104 including the sheath 134 around at least the catheter tube 114 of the intermittent catheter 102.

The uncapping step includes uncapping the cap 110 capping the proximal opening 112 of the proximal piece 106 of the intermittent catheter 102. A set forth above, the cap 110 is coupled to the proximal piece 106 by the living hinge 120.

The method further includes a foil-removing step. The foil-removing step includes removing the foil seal 150 from the distal opening 138 of the distal piece 130 of the catheter housing 104 before the catheter tube-exposing step.

The catheter tube-exposing step includes exposing an insertable portion of the catheter tube 114, which is effectuated by proximally sliding the distal piece 130 of the catheter housing 104 over the catheter tube 114. Proximally sliding the distal piece 130 of the catheter housing 104 over the catheter tube 114 in accordance with the catheter tube-exposing step includes sliding the distal piece 130 to the internal piece 132 of the catheter housing 104 to form the two-piece handle 144 for inserting the catheter tube 114 into the urethra. The distal portion 146 of the sheath 134 collapses between the distal piece 130 and the internal piece 132 when the distal piece 130 is slid to the internal piece 132. The catheter tube-exposing step further includes proximally sliding the internal piece 132 of the catheter housing 104 or the two-piece handle 144 over the catheter tube 114 to the proximal piece 106 of the intermittent catheter 102. The proximal portion 148 of the sheath 134 collapses between the internal piece 132 and the proximal piece 106 when the internal piece 132 or the two-piece handle 144 is slide to the proximal piece 106.

The catheter tube-inserting step includes inserting the catheter tube 114 into a urethra.

The urine-voiding step includes voiding urine from a bladder upon proper placement of the intermittent catheter 102 therein.

The method further includes a catheter tube-removing step, a capping step, and a catheter tube-covering step.

The catheter tube-removing step includes removing the catheter tube 114 from the urethra after the urine-voiding step.

The capping step includes capping the proximal opening 112 of the proximal piece 106 with the cap 110 after the catheter tube-removing step.

The catheter tube-covering step includes covering the insertable portion of the catheter tube 114 by distally sliding the distal piece 130 of the catheter housing 104 over the catheter tube 114.

The method can further include a joining step. The joining step includes inserting the ribbed portion 136 of the distal piece 130 of the catheter housing 104 into the receptacle 122 in the proximal portion of the cap 110 by bending proximal and distal ends of the intermittent-catheter assembly 100 toward each other. The joining step forms the after-use storage state of the intermittent-catheter assembly 100 configured to prevent urine leakage from the intermittent-catheter assembly 100 after the urine-voiding step.

The method further can further include a catheter assembly-storing step. The catheter assembly-storing step includes placing the intermittent-catheter assembly 100 in the after-use storage state thereof into the carrying case 1200 or another container for future disposal of the intermittent-catheter assembly 100.

Methods of the intermittent-catheter assembly 200 of FIGS. 13-17 and 19 include methods of using the intermittent-catheter assembly 200. For example, a method of the intermittent-catheter assembly 200 includes, in some embodiments, a catheter-obtaining step, a flange-removing step, a catheter tube-exposing step, a catheter tube-inserting step, and a urine-voiding step.

The catheter-obtaining step includes obtaining the intermittent-catheter assembly 200 in the packaged state thereof. As set forth above, the intermittent-catheter assembly 200 includes the catheter housing 204 including the sheath 234 around at least the catheter tube 214 of the intermittent catheter, wherein the flange 216 is ouroborosly inserted in the receptacle 222 in the packaged state of the intermittent-catheter assembly 200. Notably, the intermittent-catheter assembly 200 in the packaged state thereof can be further packaged in the carrying case with about a day's supply of other intermittent-catheter assemblies.

The flange-removing step includes removing the flange 216 in the proximal portion of the proximal piece 206 of the intermittent catheter 202 from the flared receptacle 222 of the distal portion of the distal piece 230 of the catheter housing 204.

The catheter tube-exposing step includes exposing the insertable portion of the catheter tube 214 including proximally sliding the distal piece 230 over the catheter tube 214 toward the proximal piece 206. The sheath 234 collapses between the distal piece 230 and the proximal piece 206 with the sliding of the distal piece 230 over the catheter tube 214, thereby unsheathing the catheter tube 214. Notably, proximally sliding the distal piece 230 over the catheter tube 214 toward the proximal piece 206 forms a two-piece handle between the proximal and distal pieces 206 and 230 for the catheter tube-inserting step.

The catheter tube-inserting step includes inserting the catheter tube 214 into a urethra.

The urine-voiding step includes voiding urine from a bladder upon proper placement of the intermittent catheter 202 therein.

The method further includes a catheter tube-removing step, a catheter tube-resheathing step, a catheter assembly-bending step, and a storage state-forming step.

The catheter tube-removing step includes removing the catheter tube 214 from the urethra after the voiding of the urine from the bladder.

The catheter tube-resheathing step includes covering the insertable portion of the catheter tube 214 with the sheath 234 by distally sliding the distal piece 230 over the catheter tube 214, thereby resheathing the catheter tube 214.

The catheter assembly-bending step includes bending proximal and distal portions of the intermittent-catheter assembly 200 toward each other.

The storage state-forming step includes ouroborosly inserting the flange 216 of the proximal piece 206 into the receptacle 222 of the distal piece 230, thereby forming an after-use storage state of the intermittent-catheter assembly 200. The after-use storage state of the intermittent-catheter assembly 200 is configured to prevent urine leakage from the intermittent-catheter assembly 200 after the voiding of the urine from the bladder.

The method can further include a storing step. The storing step includes placing the intermittent-catheter assembly 200 in the after-use storage state thereof into the multiple-use hard-shell carrying case for future disposal of the intermittent-catheter assembly 200. The intermittent-catheter assembly 200 in the after-use storage state thereof can be stored of packaged in the carrying case with the day's supply of other intermittent-catheter assemblies set forth above.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An intermittent-catheter assembly, comprising:
an intermittent catheter including:
a proximal piece including a neck; and
a catheter tube fluidly connected to the proximal piece; and
a catheter housing coupled to the intermittent catheter, the catheter housing comprising:
a distal piece configured to proximally slide over the catheter tube, wherein a distal portion of the distal piece includes a flared receptacle configured to ouroborosly accept insertion of a flange in a proximal portion of the proximal piece; and
a collapsible sheath including a distal portion coupled to the distal piece and a proximal portion coupled to the neck of the proximal piece of the intermittent catheter, an entirety of the catheter tube disposed in the catheter housing in a packaged state of the intermittent-catheter assembly.

2. The intermittent-catheter assembly of claim 1, wherein each piece of the distal piece and the proximal piece includes a medial portion having circumferential ridges configured for gripping the distal and proximal pieces when inserting the flange into the receptacle.

3. The intermittent-catheter assembly of claim 2, wherein the circumferential ridges of the distal piece are also configured for gripping the distal piece when sliding the distal piece over the catheter tube toward the proximal piece to unsheath the catheter tube or the distal piece over the catheter tube away from the proximal piece to resheath the catheter tube.

4. The intermittent-catheter assembly of claim 1, wherein the receptacle includes an inner lip and the flange includes an integrated gasket, the inner lip and the gasket configured to form a fluidly tight seal when the flange is inserted into the receptacle.

5. The intermittent-catheter assembly of claim 1, wherein the flange inserted into the receptacle forms the packaged state of the intermittent-catheter assembly, the packaged state of the intermittent-catheter assembly configured to retain a hydrophilic coating over the catheter tube, retain a moisture content of the hydrophilic coating, and maintain sterility of the intermittent catheter prior to use thereof.

6. The intermittent-catheter assembly of claim 1, wherein the flange inserted into the receptacle after use of the intermittent catheter forms an after-use storage state of the intermittent-catheter assembly, the after-use storage state of the intermittent-catheter assembly configured to prevent urine leakage from the intermittent-catheter assembly of any residual urine present in the intermittent catheter after voiding urine therewith.

7. The intermittent-catheter assembly of claim 1, wherein the catheter tube includes a plurality of eyelets proximate a catheter tip, the eyelets in fluid communication with a proximal opening of the proximal piece.

8. A method of using an intermittent-catheter assembly, comprising:
obtaining the intermittent-catheter assembly in a packaged state thereof, the intermittent-catheter assembly including a catheter housing including a sheath around at least a catheter tube of an intermittent catheter;
uncapping a cap capping a proximal opening of a proximal piece of the intermittent catheter, the cap coupled to the proximal piece by a living hinge;
exposing an insertable portion of the catheter tube including proximally sliding a distal piece of the catheter housing over the catheter tube and lubricating the catheter tube with a lubricant or lubricating sponge in a chamber or receptacle of the distal piece;
inserting the catheter tube into a urethra; and
voiding urine from a bladder upon proper placement of the intermittent catheter therein.

9. The method of claim 8, wherein the intermittent-catheter assembly in the packaged state thereof is further packaged in a package of intermittent-catheter assemblies providing about a day's supply thereof.

10. The method of claim 8, further comprising removing a foil seal from a distal opening of the distal piece before exposing the distal portion of the catheter tube.

11. The method of claim 8, wherein proximally sliding the distal piece over the catheter tube includes sliding the distal piece to an internal piece of the catheter housing to form a two-piece handle for inserting the catheter tube into the urethra, a distal portion of the sheath collapsing between the distal piece and the internal piece with the sliding of the distal piece to the internal piece.

12. The method of claim 11, wherein exposing the insertable portion of the catheter tube further includes proximally sliding the internal piece of the catheter housing or the two-piece handle over the catheter tube to the proximal piece of the intermittent catheter, a proximal portion of the sheath collapsing between the internal piece and the proximal piece with the sliding of the internal piece or the two-piece handle to the proximal piece.

13. The method of claim 8, further comprising:
removing the catheter tube from the urethra after the voiding of the urine from the bladder;
capping the proximal opening of the proximal piece with the cap; and
covering the insertable portion of the catheter tube by distally sliding the distal piece over the catheter tube.

14. The method of claim 13, further comprising inserting a ribbed portion of the distal piece into a receptacle in a proximal portion of the cap by bending proximal and distal ends of the intermittent-catheter assembly toward each other, the inserting of the distal piece into the receptacle of the cap forming an after-use storage state of the intermittent-catheter assembly configured to prevent urine leakage from the intermittent-catheter assembly after voiding urine from the bladder.

15. The method of claim 14, further comprising placing the intermittent-catheter assembly in the after-use storage state thereof into a multiple-use hard-shell carrying case for future disposal of the intermittent-catheter assembly.

16. A method of using an intermittent-catheter assembly, comprising:
obtaining the intermittent-catheter assembly in a packaged state thereof, the intermittent-catheter assembly including a catheter housing including a sheath around at least a catheter tube of an intermittent catheter;
removing a flange in a proximal portion of a proximal piece of the intermittent catheter from a flared receptacle of a distal portion of a distal piece of the catheter housing;
exposing an insertable portion of the catheter tube including proximally sliding the distal piece over the catheter tube toward the proximal piece, the sheath collapsing between the distal piece and the proximal piece with the sliding of the distal piece over the catheter tube, thereby unsheathing the catheter tube;
inserting the catheter tube into a urethra; and
voiding urine from a bladder upon proper placement of the intermittent catheter therein.

17. The method of claim 16, wherein the flange is ouroborosly inserted in the receptacle in packaged state of the intermittent-catheter assembly.

18. The method of claim 16, wherein proximally sliding the distal piece over the catheter tube toward the proximal piece forms a two-piece handle between the distal and proximal pieces for inserting the catheter tube into the urethra.

19. The method of claim 16, further comprising:
removing the catheter tube from the urethra after the voiding of the urine from the bladder;
covering the insertable portion of the catheter tube with the sheath by distally sliding the distal piece over the catheter tube, thereby resheathing the catheter tube;
bending proximal and distal portions of the intermittent-catheter assembly toward each other; and
ouroborosly inserting the flange of the proximal piece into the receptacle of the distal piece, thereby forming an after-use storage state of the intermittent-catheter assembly configured to prevent urine leakage from the intermittent-catheter assembly after the voiding of the urine from the bladder.

20. The method of claim 19, further comprising placing the intermittent-catheter assembly in the after-use storage state thereof into a multiple-use hard-shell carrying case for future disposal of the intermittent-catheter assembly.

21. The method of claim 20, wherein the intermittent-catheter assembly in the packaged or after-use storage state thereof is further packaged in the carrying case with about a day's supply of other intermittent-catheter assemblies.
